(19) United States
(12) Patent Application Publication
(10) Pub. No.: US 2016/0029994 A1
(43) Pub. Date: Feb. 4, 2016

(54) STETHOSCOPE

(71) Applicant: Wonsuk CHOI, (US)

(72) Inventor: Wonsuk CHOI, Seongnam-si (KR)

(21) Appl. No.: 14/421,235

(22) PCT Filed: Aug. 8, 2014
(86) PCT No.: PCT/KR2014/007377

§ 371 Date: Feb. 12, 2015

(30) Foreign Application Priority Data
Oct. 24, 2013 (KR) 10-2013-0126930

(51) Int. Cl. A61B 7/02; A61B 7/00

(52) U.S. Cl. CPC A61B 7/026; A61B 7/003

(57) ABSTRACT

A stethoscope according to exemplary embodiments of the inventive concept includes a chestpiece, a first connecting tube connected to the chest piece, ear pieces for a doctor connected to the first connecting tube for listening to the sound transmitted from the chestpiece, a sound collecting plate for transmitting the doctor's sound to a patient, a second connecting tube connected to the sound collecting plate, and ear pieces for a patient connected to the second connecting tube for transmitting the doctor's sound to the patient.

### TECHNICAL FIELD

Example embodiments of inventive concepts relate to a stethoscope, and more particularly, to a stethoscope informing the result of treatment to patients who have low auditory ability effectively.

### BACKGROUND ART

A stethoscope is a medical device for listening to internal sounds of human body. It is often used to listen to lung and heart sounds. It is also used to listen to intestines and blood flow in arteries and veins. Since its invention in 1819, the stethoscope has been used for listening to internal sounds of human body as an aid in the diagnosis of certain diseases or health conditions. Prior to its invention, a doctor listened to internal sounds of human body by placing his ear to patient's chest or elsewhere next to the patient's body.

Conventional stethoscopes includes a sound receiving member (or chestpiece) which is configured for placement against the surface of a patient's skin to capture internal body sounds such as those emanating from the heart, lungs, blood vessels or the like. Traditionally, there have been two types of sound receiving members provided with conventional stethoscopes, the bell type and the diaphragm type. The bell type of sound receiving member is typically used to pick up low-pitched sounds while the diaphragm type picks up high-pitched sounds. Doctors may sometimes use a stethoscope having a combination bell/diaphragm sound receiving member. The diaphragm sound receiving member includes a membrane, or diaphragm, that selectively vibrates to sounds (e.g., a heart sound) generated within a heart frequency range from within the body to amplify those sounds. Sound entering the bell type sound receiving member or the diaphragm sound receiving member is transmitted through tubing connected to a headset having a pair of earpieces configured to fit within the ears of the physician, nurse or other trained healthcare professional.

While conventional stethoscopes have been effective in checkup, they have problems in transmitting the result of checkup or doctor's requests for checkup to a patient, if the patient has low auditory ability. As a result, the checkup time becomes longer, and even the checkup may not be performed effectively.

### DETAILED DESCRIPTION OF THE INVENTION

### Technical Goal of the Invention

According to an exemplary embodiment, the inventive concept provides a pair of earpieces for patients and sound collecting plate for doctors for transmitting the result of checkup or the requests for checkup effectively.

### Technical Solution of the Invention

According to an exemplary embodiment of the inventive concept, a stethoscope includes a chestpiece for checkup a patient, a first connecting tube connected to the chestpiece, a pair of earpieces for a doctor connected to the first connecting tube to listen to the sounds transmitted from the chestpiece, a sound collecting plate for transmitting a doctor's sound to a patient, a second connecting tube connected to the sound collecting plate, and a pair of earpieces for a patient connected to the second connecting tube for transmitting the doctor's sound to the patient.

The chestpiece of the stethoscope may include a diaphragm at one side and a bell at the other side.

The stethoscope may further include a first binaural formed between the earpieces for a doctor and the first connecting tube. The first binaural may be formed on both sides connected to the first connecting tube divided into bidirectional ways.

The stethoscope may further include a connecting bridge connecting the first connecting tube divided into bidirectional ways.

The stethoscope may further include a third connecting tube wrapping the first connecting tube and the second connecting tube.

The stethoscope may further include an adjusting unit for adjusting the lengths of the first connecting tube and the second connecting tube that are movable.

The stethoscope may further include a second binaural formed between the ear pieces for a patient and the second connecting tube.

### Effect of the Invention

According to the inventive concept, a stethoscope may be effective for communicating smoothly between a doctor and a patient who has low auditory ability.

### BRIEF DESCRIPTION OF THE DRAWINGS

FIG. 1 is a perspective view of a stethoscope according to an exemplary embodiment of the inventive concept;

FIG. 2 is a diagram illustrating a structure of a first connecting tube and a second connecting tube; and

FIG. 3 is a diagram illustrating another structure of a first connecting tube and a second connecting tube.

### BEST MODE FOR CARRYING OUT THE INVENTION

Various example embodiments will be described more fully hereinafter with reference to the accompanying drawings, in which some example embodiments are shown. Inventive concepts may, however, be embodied in many different forms and should not be construed as limited to the example embodiments set forth herein.

In the drawings, the sizes and relative sizes of layers and areas may be exaggerated for clarity, and the sizes are just examples not restrictions. Like numerals refer to like elements throughout.

The example embodiments of the present invention explain idealized example embodiments of the inventive concept in detail. Therefore, various transformations of diagrams may be performed. The example embodiments are not restricted to the diagrams and include transformation of form during manufacturing.

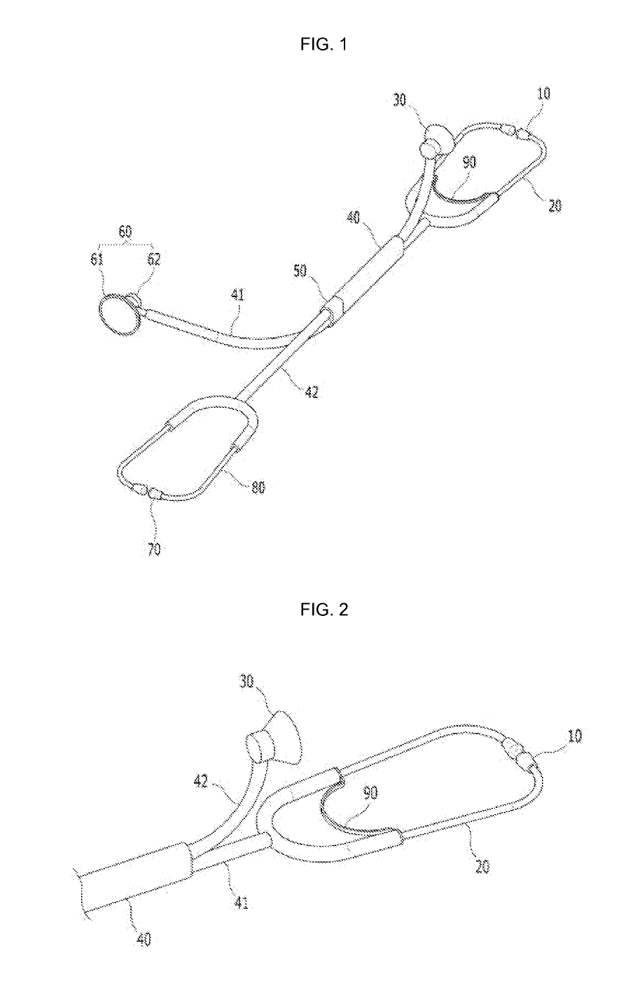

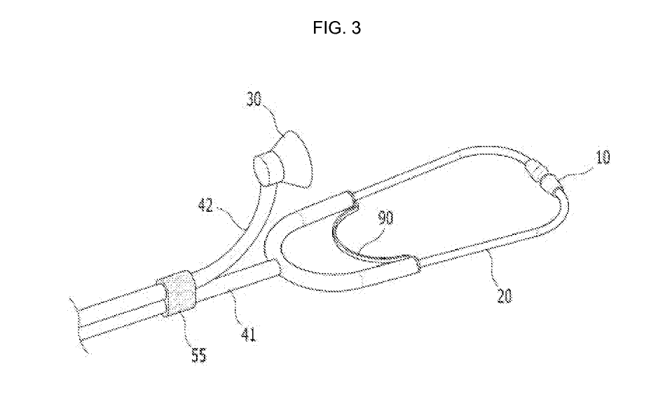

Hereinafter, according to exemplary embodiments of the inventive concept, a stethoscope will be described referring to FIGS. 1 through 3.

As shown in FIG. 1, the stethoscope includes a chestpiece 60 for diagnosing a patient.

The chestpiece 60 is required to listen to internal sounds of a patient's body that are the sounds from intestines and blood flow in arteries and veins including heart sound and breath sound to check whether the body is in a regular condition or not. Also, the stethoscope is used to listen to the sounds from artery in the arm when taking the patient's pulse.

The chestpiece 60 generally includes a diaphragm on one side and a bell on the other side. The diaphragm 61 is a membrane that vibrates according to the sound waves in the wavelength of 100 Hz through 1 KHz, that is, usually the sounds from lungs and intestines.

The bell 62 is caved in like a bell shape through which the wavelength of 20 Hz through 200 Hz, usually the sounds from opening and shutting of heart valves or back flowing or swirling of blood flow, may be heard. The sounds are relatively low pitch. Requesting to stop breathing in treatment is for listening to the exact heart sound.

The way to listen to small-scale sounds is important in diagnosis through a stethoscope, because small-scale sounds come from heart, lungs, or intestines are gathered through sound collecting parts and transmitted to the listener. The bell 62 is used to listen to the sounds of low frequencies, and the diaphragm 61 is used to listen to the sounds of relatively high pitch and wider wavelength. The difference is caused by the structure of sound collecting parts. The sound generated in the body vibrates air without a membrane that vibrates in the bell, thus, small-scale sounds may be heard. By the way, in case of the diaphragm 61, once vibrations from the skin are transmitted to a plastic membrane that vibrates, and the vibrations of the plastic membrane vibrate air to transmit the sound. During that process, small-scale sounds are absorbed to the membrane that vibrates itself and may not be transmitted further, accordingly, the diaphragm 61 requires vibrations over a certain level.

Listening to the internal sounds through a stethoscope is that the internal sounds vibrate the membrane of the stethoscope, and then the vibrations are transmitted to the doctor's ear. Sound is a wave transmitted through a medium, wherein the progress direction of the wave and the vibrating direction of the medium are same as a longitudinal wave.

The stethoscope according to exemplary embodiments of the inventive concept includes a first connecting tube 41 connected to the chestpiece and ear pieces 10 for a doctor to listen to the sounds transmitted from the chestpiece 60, which are connected to the first connecting tubes 41. The ear pieces are inserted to the ears in directions of the external auditory meatus while placing a little bit bent side to the front and sticking to the skin to listen to the sound well. The ear pieces are for transmitting the sounds lastly in the listener's ear, thus, they need to fit earholes adequately and unpainful for wearing for a long time, and material harmless to human body is used, because they contact human body.

The first connecting tube 41 is like a path way for transmitting the sound source gathered in the sound gcollecting plate to ears.

A tube type stethoscope may miss the high wavelength band structurally, so some stethoscopes may have short connecting tubes to avoid the same.

The stethoscope according to exemplary embodiments of the inventive concept further includes a sound collecting plate 30 for transmitting the doctor's sound to the patient, a second connecting tube 42 connected to the sound collecting plate 30, and ear pieces for a patient 70 connected to the second connecting tube for transmitting the doctor's sound to the patient.

The sound collecting plate 30 is for transmitting the doctor's sound to the patient who has low auditory ability, and it is like a chestpiece. The sound collecting plate may be made of a chestpiece, and any devices having a function of collecting sound may be applicable.

The sounds gathered through the sound collecting plate are transmitted to the earpieces for a patient 70 traveling through the second connecting tube 42. The sounds may be transmitted to the patient who has low auditory ability through the sound collecting plate effectively, resulting in solving the problem that the patient may not understand what the doctor says with his voice without the sound collecting plate.

The chestpiece 60 of the stethoscope includes a diaphragm 61 on one side and a bell 62 on the other side. The diaphragm and the bell are identical those explained above.

The stethoscope further includes a first binaural 20 formed between the earpiece for a doctor and the first connecting tube. The binaural are worn on the ears and transmits the sound comes from the connecting tube to the earpiece. Usually it is made of reinforced aluminum, steel, or copper, but it may be made of plastic as occasion demands.

The first binaural may be formed on both sides connected to the first connecting tube divided into bidirectional ways, and the stethoscope may further include a connecting bridge 90 connecting the first connecting tube divided into bidirectional ways.

The stethoscope may further include a third connecting tube 40 wrapping the first connecting tube and the second connecting tube.

The first connecting tube and the second connecting tube are preferred to be stick together for easy of treatment. At this time, the third connecting tube 40 wrapping the first connecting tube and the second connecting tube is prepared as shown in FIG. 2.

The stethoscope may further include an adjusting unit 50 adjusting the length of the first connecting tube and the second connecting tube that are movable. Two connecting tubes are prepared, accordingly, it is necessary to adjust the length of each connecting tube that is movable as occasion demands. At this time, the adjusting unit 50 is used for adjusting the lengths of the first connecting unit and the second connecting unit that are movable for the doctor to checkup the patient effectively.

The stethoscope may further include a second binaural 80 between the earpieces for a patient and the second connecting tube. The function and the material of the second binaural are identical to those of the first binaural.

While the present invention has been particularly shown and described with reference to exemplary embodiments thereof, it will be understood that various changes in form and details may be made therein without departing from the spirit and scope of the following claims.

### INDUSTRIAL USABILITY

The present invention is applicable to a stethoscope to be used in checking up in hospital, or the like.

What is claimed is:

1. A stethoscope comprising:
a chestpiece for diagnosing a patient;
a first connecting tube connected to the chestpiece;
ear pieces for a doctor connected to the first connecting tube for listening to the sound transmitted from the chestpiece;
a sound collecting plate for transmitting the doctor's sound to a patient;
a second connecting tube connected to the sound collecting plate; and
ear pieces for a patient connected to the second connecting tube for transmitting the doctor's sound to the patient.

2. The stethoscope of claim 1, wherein the chestpiece includes a diaphragm on one side and a bell on the other side.

3. The stethoscope of claim 1, wherein a first binaural is included between the ear pieces for a doctor and the first connecting tube.

4. The stethoscope of claim 3, wherein the first binaural are formed on both sides connected to the first connecting tube divided into bidirectional ways.

5. The stethoscope of claim 4, wherein a connecting bridge is prepared for connecting the first connecting tube divided into bidirectional ways.

6. The stethoscope of claim 1 further includes a third connecting tube wrapping the first connecting tube and the second connecting tube.

7. The stethoscope of claim 1 further includes an adjusting unit for adjusting the lengths of the first connecting tube and the second connecting tube that are movable.

8. The stethoscope of claim 1 further includes a second binaural formed between the earpieces for a patient and the second connecting tube.
